(19) United States
(12) Patent Application Publication
(10) Pub. No.: US 2014/0316398 A1
(43) Pub. Date: Oct. 23, 2014

(54) SYSTEMS AND METHODS RELATED TO SELECTIVE HEATING OF CRYOGENIC BALLOONS FOR TARGETED CRYOGENIC NEUROMODULATION

(71) Applicants: Brian Kelly, Ballybrit (IE); Gary Kelly, Windsor (IE); Barry Mullins, Wicklow (IE); Fiachra Sweeney, Ballybrit (IE)

(72) Inventors: Brian Kelly, Ballybrit (IE); Gary Kelly, Windsor (IE); Barry Mullins, Wicklow (IE); Fiachra Sweeney, Ballybrit (IE)

(21) Appl. No.: 14/114,566

(22) PCT Filed: Apr. 27, 2012
(86) PCT No.: PCT/US12/35482

§ 371 Date: Jun. 12, 2014

Related U.S. Application Data
(60) Provisional application No. 61/572,289, filed on Apr. 29, 2011.

(51) Int. Cl. A61B 18/02

(52) U.S. Cl. CPC A61B 18/02; A61B 2018/00041
USPC 606/24

(57) ABSTRACT

Systems and methods related to selective heating of cryogenic balloons for targeted cryogenic neuromodulation are disclosed herein. A cryotherapeutic device configured in accordance with a particular embodiment of the present technology can include an elongated shaft having a proximal portion and a distal portion. The shaft can be configured to locate the distal portion in a vessel. The cryotherapeutic device can further include a cryoballoon extending from the distal portion and a plurality of heating elements arranged about the cryoballoon. The plurality of heating elements can be individually controlled to selectively deliver heat to tissue of a wall of the vessel proximate the outer surface of the cryoballoon.

### CROSS REFERENCE TO RELATED APPLICATION

This disclosure claims the benefit of U.S. Provisional Application No. 61/572,289, filed Apr. 29, 2011, which is incorporated herein by reference in its entirety.

### TECHNICAL FIELD

The present technology relates generally to cryotherapeutic systems and methods. In particular, several embodiments are directed to systems and methods for cryogenically cooling a targeted area of an inner surface of an anatomical vessel.

### BACKGROUND

Cryotherapy can be a useful treatment modality in a wide range of catheter-based interventional procedures. For example, cryotherapeutic cooling can be used to modulate nerves or affect other tissue proximate anatomical vessels (e.g., blood vessels, other body lumens, or other areas in the body). This can reduce undesirable neural activity to achieve therapeutic benefits. Catheter-based neuromodulation utilizing cryotherapy can be used, for example, to modulate nerves and thereby reduce pain, local sympathetic activity, systemic sympathetic activity, associated pathologies, and other conditions. Furthermore, cryotherapy can be used, for example, for ablating tumors and treating stenosis. In some cryotherapeutic procedures, it can be useful to deliver cryotherapy via a balloon that can be expanded within an anatomical vessel. Such balloons can be operatively connected to extracorporeal support components (e.g., refrigerant supplies). As the applicability of cryotherapy for surgical intervention continues to expand, there is a need for innovation in the associated devices, systems, and methods. Such innovation has the potential to further expand the role of cryotherapy as a tool for improving the health of patients.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present technology.

FIG. 1 is a partially schematic isometric view of a common location of neural fibers proximate an artery.

FIG. 2 is a side view of a cryotherapy catheter having a cryoballoon with electrical heating elements configured in accordance with an embodiment of the present technology.

FIG. 2A is a cross-sectional view taken along line A-A of FIG. 2.

FIG. 2B is a cross-sectional view taken along line B-B of FIG. 2.

FIG. 2C is a cross-sectional view taken along line C-C of FIG. 2.

FIGS. 3A-3G illustrate various arrangements of electrical heating elements on a cryoballoon configured in accordance with embodiments of the present technology.

FIG. 4 is a cross-sectional view of the cryoballoon of FIG. 2, wherein a polymer coating is utilized to couple the heating elements to the cryoballoon.

FIG. 5 is a cross-sectional view of a cryoballoon having heating elements configured in accordance with another embodiment of the present technology, wherein an outer balloon or sheath is utilized to couple the heating elements to the cryoballoon.

FIG. 6 is a side view of a cryotherapy catheter having a cryoballoon with microtubes that receive heated fluid in a circulating manner in accordance with an embodiment of the present technology.

FIG. 6A is a cross-sectional view taken along line A-A of FIG. 6.

FIG. 6B is a cross-sectional view taken along line B-B of FIG. 6.

FIG. 6C is a cross-sectional view taken along line C-C of FIG. 6.

FIG. 6D is a cross-sectional view taken along line A-A of FIG. 6 according to another embodiment of the present technology.

FIG. 6E is a cross-sectional view taken along line C-C of FIG. 6 according to another embodiment of the present technology.

FIGS. 7A-7G illustrate various arrangements of microtubes on a cryoballoon configured in accordance with embodiments of the present technology.

FIG. 8 is a side view of a cryotherapy catheter having a cryoballoon with microtubes that receive heated fluid in a non-circulating manner in accordance with a further embodiment of the present technology.

FIG. 8A is a cross-sectional view taken along line A-A of FIG. 8.

FIG. 9 is a side view of a cryotherapy catheter having a cryoballoon with microtubes that receive blood flow from a vessel lumen in accordance with an embodiment of the present technology.

FIG. 9A is a cross-sectional view taken along line A-A of FIG. 9.

FIG. 10 is a schematic side view of the cryoballoon of FIG. 9 disposed within a vessel.

FIG. 11 is a cross-sectional view of a cryoballoon deployed in a vessel and having insulative microtubes configured in accordance with another embodiment of the present technology.

FIG. 12 is a cross-sectional view of a cryoballoon deployed in a vessel and having solid conductive microtubes configured in accordance with yet another embodiment of the present technology.

### DETAILED DESCRIPTION

Specific embodiments of the present technology are now described with reference to the figures, wherein like reference numbers indicate identical or functionally similar elements. The terms “distal” and “proximal” are used in the following description with respect to a position or direction relative to the treating clinician. “Distal” and “distally” refer to positions distant from or in a direction away from the clinician. “Proximal” and “proximally” refer to positions near or in a direction toward the clinician.

The following detailed description discloses specific examples of the present technology, but it is not intended to limit the present technology or the application and uses of the present technology. For example, although the description discloses the present technology in the context of treatment of blood vessels, such as coronary, carotid and renal arteries, the present technology may also be used in other body passageways or tissues where it is deemed useful. Furthermore, there is no intention to be bound by any expressed or implied theory presented herein.

In recent years, ablation of tissue has been used to modulate neural fibers that contribute to renal function. Ablation may be accomplished in various ways, including delivery of radio frequency (RF) energy, other suitable heating energies, or cryotherapy. Modulation of renal nerves is expected to be useful in treating a variety of renal, cardio-renal, and other diseases including heart failure, renal disease, renal failure, hypertension, contrast nephropathy, arrhythmia, and myocardial infarction. Furthermore, renal neuromodulation is expected to reduce renal sympathetic nervous activity, which can increase removal of water and sodium from the body and return renin secretion to more normal levels. Normalized renin secretion can cause blood vessels supplying the kidneys to assume a steady state level of dilation and constriction corresponding to adequate renal blood flow.

In neuromodulation procedures, it may be desirable to perform circumferential ablation that extends continuously about a full 360° of the circumference of an anatomical vessel to positively affect a medical condition. For example, in the treatment of atrial fibrillation or other arrhythmia, a circumferential treatment may be achieved by forming a circumferential lesion that is continuous completely about a normal cross-section of the pulmonary vein to disrupt aberrant electrical signals. In the treatment of heart failure, a circumferential treatment may be achieved by forming a similar continuous circumferential lesion that is continuous completely about a normal cross-section of a renal artery to reduce renal sympathetic neural activity. However, in some cases, it can be desirable to reduce structural changes to a blood vessel and avoid a circumferential ablation lesion along a single radial plane or cross-section of a blood vessel. Partial circumferential, non-continuous, or helical ablation are expected to be effective to treat a variety of renal, cardio-renal, and other diseases including those listed herein with fewer structural changes to vessels than fully circumferential, continuous, and non-helical ablation.

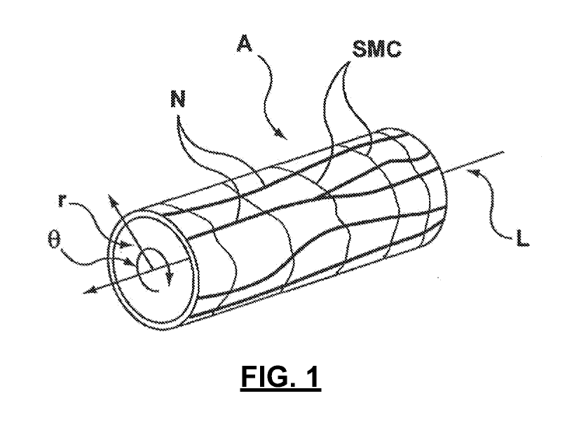

FIG. 1 illustrates a common anatomical arrangement of neural structures relative to body lumens or vascular structures (e.g., arteries). Neural fibers N generally may extend longitudinally along a lengthwise or longitudinal dimension L of an artery A about a relatively small range of positions along the radial dimension r, often within the adventitia of the artery. The artery A has smooth muscle cells SMC that surround the arterial circumference and generally spiral around the angular dimension θ of the artery, also within a relatively small range of positions along the radial dimension r. The smooth muscle cells SMC of the artery A accordingly have a lengthwise or longer dimension generally extending transverse (i.e., nonparallel) to the lengthwise dimension of the blood vessel.

Neuromodulation can refer to inhibiting, reducing, and/or blocking neural communication along neural fibers (i.e., efferent and/or afferent nerve fibers), and may be accomplished by ablating tissue through the use of an ablation catheter. As used herein, the term ablation includes the creation of scar tissue or a lesion that blocks or disrupts nerve conduction. In embodiments hereof, freezing temperatures or cryotherapy can be utilized to thermally damage or ablate target tissue of an artery to achieve neuromodulation of the target neural fibers. As compared to ablation via RF energy, cryotherapy typically uses less power to achieve neuromodulation.

The present technology relates to devices, systems, and methods for protecting non-target tissue from cryogenic ablation by a cryotherapy catheter in order to provide partial circumferential (i.e., ablation extending around less than 360° of a vessel wall) or non-continuous circumferential cryoablation. In order to form partial or non-continuous circumferential ablations, a cryoballoon can be configured to deliver cryotherapeutic cooling to focused target regions of tissue to be treated, and non-targeted tissue can be protected from ablation by one or more heating elements that protect or shield the non-targeted tissue from ablation. As will be explained in more detail below, the heating elements may include electrical wires or electrodes that are heated via electrical current and/or microtubes that receive heated fluids.

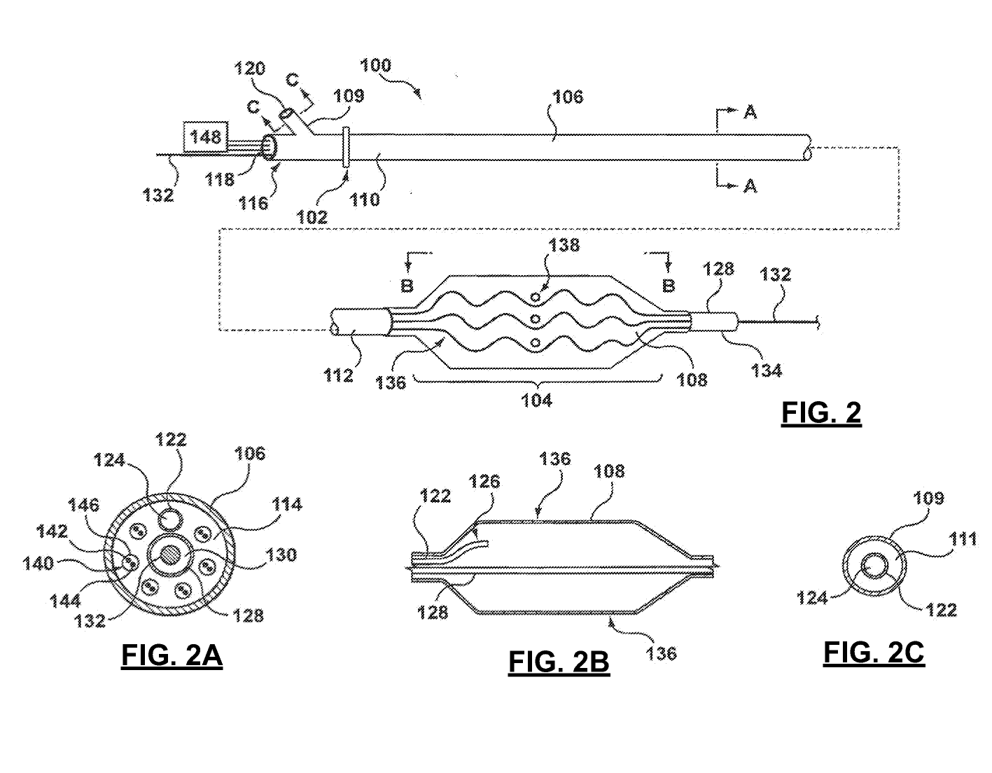

FIGS. 2, 2A, 2B, and 2C illustrate a cryotherapy balloon catheter 100 for ablating tissue to provide neuromodulation of the targeted nerves. Cryotherapy catheter 100 can include a proximal portion 102 that extends out of the patient and has a hub 116. A distal portion 104 of cryotherapy catheter 100 can be delivered to a targeted location within the vasculature and can include a cryoballoon 108, which is shown in an expanded or inflated configuration in FIG. 2, having a plurality of heating elements 136 thereon. In the embodiment shown in FIGS. 2, 2A, 2B, and 2C, catheter 100 has an over-the-wire (OTW) catheter configuration with an inner shaft 128 that defines a guidewire lumen 130 extending substantially the entire length of the catheter for accommodating a guidewire 132. Inner shaft 128 can have a proximal end (not shown) coupled to a proximal guidewire port 118 of hub 116 and a distal end 134 terminating distally of cryoballoon 108 and defining a distal guidewire port. Catheter 100 can also include a tubular component or outer shaft 106 which defines a lumen 114 and can have a proximal end 110 coupled to hub 116 and a distal end 112 coupled to cryoballoon 108.

Catheter 100 may further include a cryo-supply shaft 122 extending through outer shaft 106, cryo-supply shaft 122 defining an inflation lumen 124 and having a proximal end (not shown) coupled to hub 116 and a distal end 126 (see FIG. 2B) that terminates within cryoballoon 108. A cryo-inflation port 120 of hub 116 can be placed in fluid communication with inflation lumen 124 of cryo-supply shaft 122. Cryo-supply shaft 122 can receive and deliver a cryogenic agent, such as N₂O liquid, into cryoballoon 108 at a high pressure (e.g., 800 psi) such that there is a pressure drop when the cryogenic agent enters the interior of cryoballoon 108 and expands to a gas. The cryogenic agent may be any liquid having a boiling point colder than approximately −10° C. at atmospheric pressure, such as N₂O liquid or CO₂ liquid. During the phase change of the cryogenic agent, a cooling effect can take place because expansion of a compressed gas is an endothermic process that absorbs energy in the form of heat and thus results in cooling of the surroundings. Accordingly, as the cryogenic agent expands into gas, cryoballoon 108 expands or inflates and the outer surface of the cryoballoon cools to cryogenic temperatures operable to ablate or thermally damage tissue. For example, the temperature of cryoballoon 108 can be approximately between −5° C. and −120° C., which is expected to result in neuromodulation of neural fibers located adjacent to cryoballoon 108. As would be understood by one of ordinary skill in the art, hub 116 can provide a luer hub or other type of fitting that may be connected to a source of the cryogenic agent and may be of another construction or configuration without departing from the scope of the present technology.

As shown in the sectional view of FIG. 2B, cryo-supply shaft 122 and inner shaft 128 can extend freely through (e.g., are not bonded to) outer shaft 106 and cryoballoon 108. As noted above, a continuous supply of cryofluid can exit distal end 126 of cryo-supply shaft 122 into an interior of cryoballoon 108 to expand therein. The expanded cryogenic gas can proximally exit the interior of cryoballoon 108 via a space within lumen 114 between shafts 122, 128 and outer shaft 106. In certain embodiments, a vacuum may be utilized to draw the expanded cryogenic gas out of the catheter. The expanded cryogenic gas can travel proximally within lumen 114 of outer shaft 106 to exit or exhaust from catheter 100 via an arm 109 of hub 116. As shown in the cross-sectional view of FIG. 2C, cryotherapy shaft 122 can extend through arm 109 such that an exhaust space 111 is defined between cryo-supply shaft 122 and an inner surface of arm 109. The expanded cryogenic gas may escape through the exhaust space 111.

The multiple catheter shafts of catheter 100 (e.g., outer shaft 106, inner shaft 128, and cryo-supply shaft 122) may be formed from one or more polymeric materials, such as polyethylene, polyethylene block amide copolymer (PEBA), polyamide and/or combinations thereof (e.g., laminated, blended or co-extruded combinations). In various embodiments, inner shaft 128 may be a flexible tube of a polymeric material, such as polyethylene tubing. Optionally, outer shaft 106 or some portion thereof may be formed as a composite having a reinforcement material incorporated within a polymeric body in order to enhance strength and/or flexibility. Suitable reinforcement layers include braiding, wire mesh layers, embedded axial wires, embedded helical or circumferential wires, and the like. In one embodiment, for example, at least a proximal portion of outer shaft 106 may be formed from a reinforced polymeric tube. In addition, although catheter 100 is described herein as being constructed with various shafts extending therethrough for forming the lumens of the catheter, it will be understood by those of ordinary skill in the art that other types of catheter construction are also amendable to the present technology, such as a catheter shaft formed by multi-lumen profile extrusion. In another embodiment, catheter 100 may be modified to be of a rapid exchange (RX) catheter configuration such that inner shaft 128 extends within only the distal portion of catheter 100.

A plurality of heating elements 136 can be disposed over the outer surface of cryoballoon 108. When cryoballoon 108 expands, heating elements 136 can be positioned between cryoballoon 108 and a vessel wall to shield or prevent the cryoballoon from ablating non-targeted tissue of the vessel wall. In other embodiments, the heating elements 136 can be embedded in the cryoballoon 136 and/or positioned within the cryoballoon 136. Tissue of the vessel wall can come into contact or near-contact with cryoballoon 108 at the areas or spaces formed between heating elements 136. Accordingly, spaces between heating elements 136 can serve as areas for cryotherapy ablation, and the geometry of heating elements 136 can therefore form an ablation therapy pattern. As mentioned above, the temperature of cryoballoon 108 can be between about −5° C. and about −120° C. to induce neuromodulation of neural fibers located adjacent to cryoballoon 108. In order to shield or prevent non-targeted tissue from ablation, the temperature of heating elements 136 can be between about 5° C. and about 45° C. In one embodiment, for example, the temperature of heating elements 136 is approximately 37° C. The minimum temperature (e.g., 5° C.) of the heating elements 136 can be selected to inhibit or prevent cryogenic thermal injury or denervation that would otherwise result from the cryogenic outer surface temperature of cryoballoon 108. Further, of the maximum temperature (e.g., 45° C.) of the heating elements 136 can be selected to reduce the risk of or prevent undesired ablation of the tissue due to thermal injury or stress caused by heat. In FIG. 2, heating elements 136 each have a sinusoidal configuration and longitudinally extend along the working length of cryoballoon 108 to result in a partial circumferential ablation pattern. The working length of the cryoballoon as used herein is intended to describe the longitudinal portion of the cryoballoon which expands against and contacts the vessel wall.

In the embodiment illustrated in FIGS. 2-2C, the heating elements 136 are electrical wires or electrodes that can be heated by resistive heating, although disc electrodes, flat electrodes, and/or other electrodes are also suitable for use herein. Electrode heating elements 136 may be formed from any suitable metallic material, such as gold, platinum or a combination of platinum and iridium. An external power supply 148 can supply electrical current to heating elements 136. In one embodiment, power supply 148 may be a multi-channel radio frequency generator, such as the GENIUS® generator available from Medtronic Ablation Frontiers of Carlsbad, Calif. In order to provide a desired temperature of heating elements 136 between about 5° C. and 45° C., power supply 148 may deliver power between 0 W and 15 W. In one embodiment, power supply 148 supplies between 5 W and 10 W. In the embodiment depicted in FIG. 2, heating elements 136 include six wires or electrodes equally spaced apart along the circumference of cryoballoon 108; however, it will be apparent to one of ordinary skill in the art that the number of heating elements 136 and the spacing therebetween may be varied. In one embodiment, for example, the cryoballoon 108 can include a single heating element extending along or around the surface of the cryoballoon 108. Multiple heating elements 136 allow for various ablation patterns via a single device as described in more detail herein.

Each electrode heating element 136 can be electrically connected to power source 148 by a conductor or wire that extends through lumen 114 of outer shaft 106. Since the embodiment of FIG. 2 includes six electrode heating elements 136 (although only three are visible in the side view of FIG. 2), six corresponding bifilar or other wires 140 electrically connect a respective electrode heating element 136 to power source 148. Each electrode heating element 136 may be welded or otherwise electrically coupled to the distal end of its wire 140, and each wire 140 can extend through outer shaft 106 for the entire length of catheter 100 such that a proximal end thereof is coupled to power source 148. Accordingly, each electrode heating element 136 may be independently controllable via its respective wire 140. As such, an operator may selectively activate or deactivate a particular heating element 136 in order to vary the resulting ablation pattern. Numerous combinations of activated heating elements are possible, so a single device may be utilized to effectuate various ablation patterns.

With reference to FIG. 2A, in one embodiment, each wire 140 is a bifilar wire that includes a first conductor 142, a second conductor 144, and insulation 146 surrounding each conductor to electrically isolate them from each other. In certain embodiments, first conductor 142 may be a copper conductor, second conductor 144 may be a copper/nickel conductor, and insulation 146 may be polyimide insulation. When coupled to an electrode heating element, the two conductors of each bifilar wire 140 can function to provide power to its respective electrode and act as a T-type thermocouple for the purposes of measuring the temperature of the electrode. Temperature measurements can provide feedback to power source 148 such that the power delivered to each electrode can be automatically adjusted by the power source to achieve a target temperature. Temperature measurements can also provide an indication of the quality of the contact between the electrode and the adjacent tissue. In various embodiments, the power source 148 may display the power each electrode heating element 136 is receiving and the temperature achieved during ablation.

In another embodiment hereof, wires 140 may be single conductor wires rather than the bifilar wires described above. Each single conductor wire provides power to its respective electrode. In this embodiment, separate temperature sensors can be used to determine the temperature of the heating elements 136 and provide feedback to the power source 148.

In addition to shielding non-targeted tissue from ablation, heating elements 136 may additionally or alternatively serve to moderate and/or maintain the temperature of the cryotherapy. For example, when N₂O liquid is utilized as the cryogenic agent, the phase change of the cryogenic agent to gas may result in an outside or exterior surface of the cryoballoon 108 reaching a cryoballoon temperature in the range of −70° C. to −80° C. However, cryogenically-induced neuromodulation may be accomplished at substantially warmer temperatures (e.g., between −5° C. and −40° C.). Since heating elements 136 are disposed over cryoballoon 108, heat transfer occurs therebetween. Due to heat transfer from cryoballoon 108, the temperature at heating elements 136 may decrease, but not to a temperature that results in thermal modulation (e.g., the temperature at the heating elements can be kept above −5° C.). Heat transfer to cryoballoon 108 from heating elements 136 may be beneficial to increase the temperature of the cryogenically-cooled balloon outer surface from, e.g., −80° C., to a temperature suitable for neuromodulation, e.g., between −10° C. and −40° C. Thus, the heat transfer between the cryoballoon 108 and the heating elements 136 may help to moderate the temperature of the cryotherapy.

Catheter 100 can also include a thermocouple 138 associated with each heating element 136 for monitoring the temperature of tissue adjacent to the thermocouple 138 and/or the temperature of the outside surface of cryoballoon 108 at various locations on the device. In certain embodiments, thermocouple 138 measures an average of both the temperature of tissue adjacent to the thermocouple 138 and the temperature of the outside surface of cryoballoon 108. Thermocouples 138 and/or other temperature sensors can be coupled to the outer surface of the cryoballoon 108 in close proximity to each heating element 136. Thermocouples 138 may be utilized in regulating or moderating the outer surface temperature of cryoballoon 108. Monitoring the temperature of cryoballoon 108 via thermocouples 138 allows the operator to determine which heating elements 136 should be active. For example, if the temperature profile of the outer surface of cryoballoon 108 is not even and a particular region is colder than desired, a heating element 136 in the colder region may be activated in order to moderate the temperature thereof. Thermocouples 138 are therefore useful in maintaining a steady state surface temperature and/or are useful to achieve a variable temperature profile or gradient on the surface of cryoballoon 108 if desired.

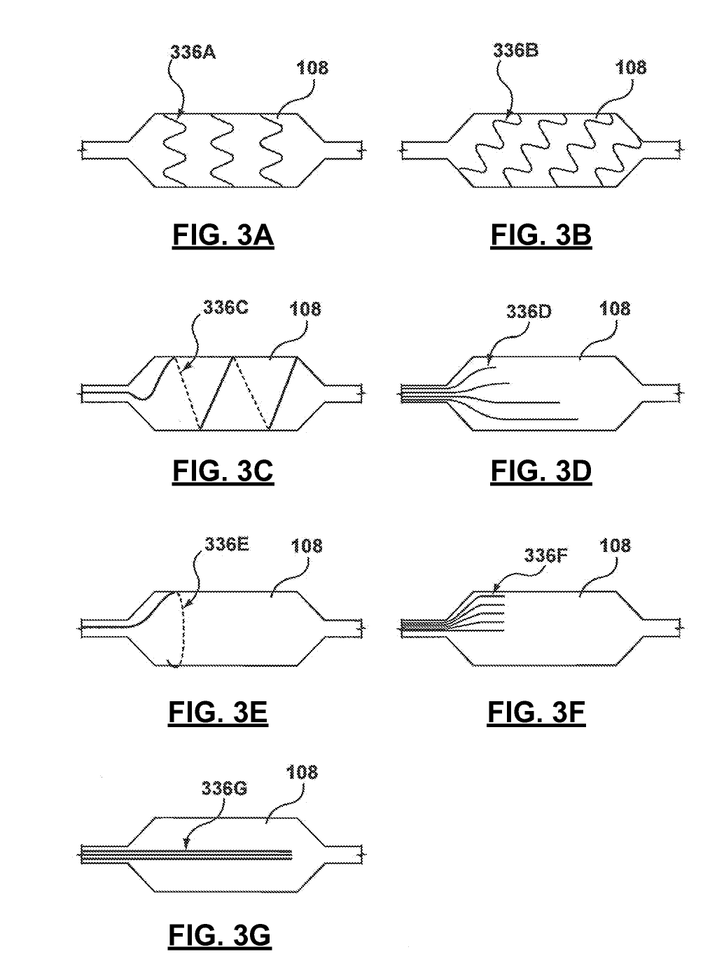

It will be apparent to those of ordinary skill in the art that various configurations of heating elements are possible in order to achieve a number of different ablation patterns. The path of heating elements 136 may extend in a spiral, a straight line, or partially around the circumference of cryoballoon 108. FIGS. 3A-3G illustrate various configurations of heating elements 336. FIG. 3A illustrates a plurality of sinusoidal heating elements 336A extending around a circumference of balloon 108, while FIG. 3B illustrates a plurality of sinusoidal heating elements 336B extending around a circumference of balloon 108 at an angle. In another embodiment, the configuration shown in FIG. 3B may be accomplished by a single sinusoidal heating element that spirals around the length of the balloon. FIG. 3C illustrates a single spiral heating element 336C extending around a circumference of balloon 108. FIG. 3D illustrates a plurality of straight heating elements 336D of differing lengths extending longitudinally along balloon 108. FIG. 3F is similar to FIG. 3D, except heating elements 336F are not of differing lengths and are restrained to a quadrant of balloon 108. Further, FIG. 3G is similar to FIG. 3D, except heating elements 336G are not of differing lengths and are restrained to a single linear or longitudinal strip of balloon 108. FIG. 3E illustrates a single heating element 336E extending partially around a circumference of balloon 108. It will be apparent to those of ordinary skill in the art that numerous patterns or configurations of the heating elements beyond those described herein may be utilized in order to cause a desired ablation pattern having a combination of targeted and non-targeted tissue of the vessel wall.

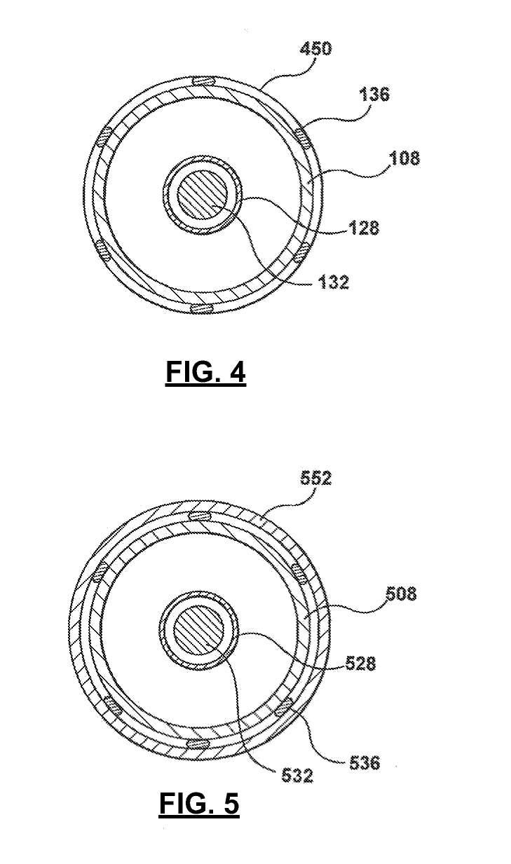

Heating elements 136 may be coupled to the cryoballoon 108 in various manners. For example, in the embodiment illustrated in FIG. 4, electrode heating elements 136 may be coupled to the balloon outer surface in the desired pattern via an adhesive or other mechanical method. The cryoballoon 108 having electrode heating elements 136 coupled thereto is then dipped into a polymer such as polyurethane or silicone. The polymer cures, resulting in a flexible coating 450 disposed over cryoballoon 108, holding electrode heating elements 136 in position on the cryoballoon. In other embodiments, the heating elements 136 may be manufactured in a specific pattern and embedded in a polymer layer that can subsequently be attached to a cryoballoon. The polymer coating 450 may be polyurethane. In another embodiment shown in FIG. 5, a cryoballoon 508 having heating elements 536 disposed thereon is surrounded by an outer balloon or sheath 552 in order to capture heating elements 536 between the outer surface of cryoballoon 508 and sheath 552.

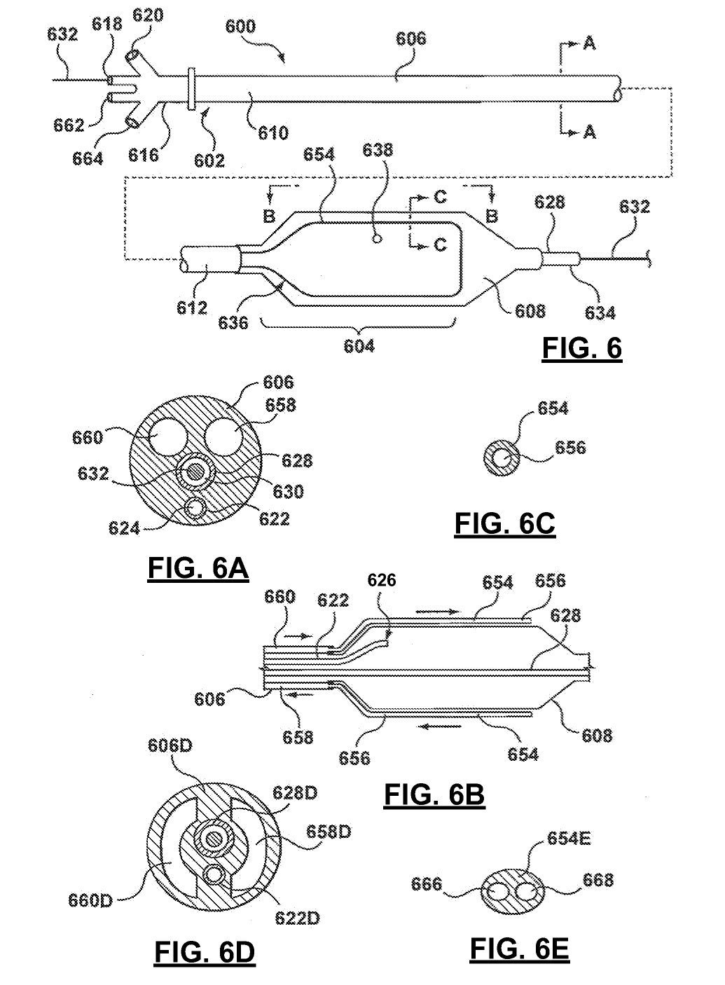

In other embodiments, the heating elements for shielding non-targeted tissue from ablation include one or more microtubes that are configured to receive heated fluids (e.g., liquids or gases). Referring to FIG. 6, a cryotherapy catheter 600 is utilized for ablating tissue to provide neuromodulation of the targeted nerves and heating elements 636 are utilized for shielding non-targeted tissue from ablation. As opposed to electrodes for protecting non-targeted tissue from ablation, the heating elements 636 of the catheter 600 are defined by at least one microtube 654 disposed over cryoballoon 608 that receives a heated fluid, such as saline, contrast media, blood, plasma, carbon dioxide, and/or oxygen. The temperature of the heated fluid within the heating elements 636 can be between about 20° C. and about 45° C. in order to provide a shielding function, and the temperature can be above about 0° C. to provide a moderating function. Cryotherapy catheter 600 can include a proximal portion 602 that extends out of the patient, and the proximal portion 602 may include a hub 616. Distal portion 604 of catheter 600 can be delivered to a targeted location within the vasculature and can include cryoballoon 608, which is shown expanded or inflated in FIG. 6. Catheter 600 can include an outer shaft 606 having a proximal end 610 coupled to hub 616 and a distal end 612 coupled to cryoballoon 608. A guidewire shaft 628 and cryo-supply shaft 664 can extend within a lumen of outer shaft 606. Similar to inner shaft 128 described above, guidewire shaft 628 defines a guidewire lumen 630 extending substantially an entire length of catheter 600 for accommodating a guidewire 632. Guidewire shaft 628 can have a proximal end (not shown) coupled to a proximal guidewire port 618 of hub 616 and a distal end 634 terminating distally of cryoballoon 608 and defining a distal guidewire port. Cryo-supply shaft 622 can define an inflation lumen 624, which may have a distal end 626 that terminates within cryoballoon 608. A cryo-inflation port 620 of hub 616 may be in fluid communication with inflation lumen 624 of cryo-supply shaft 622, and cryo-supply shaft 622 can receive and deliver a cryogenic agent (e.g., N2O liquid) into cryoballoon 608 (e.g., as described above with respect to cryo-supply shaft 122 and cryoballoon 108).

As shown in FIG. 6C, microtube 654 can be a tubular component defining a lumen 656. In one embodiment, microtube 654 has an inner diameter between about 0.025 mm (0.001 inch) to about 0.152 mm (0.006 inch). Suitable materials for microtube 654 may include polyimide, PEEK, stainless steel, and Nitinol. Microtube 654 may be coupled to the cryoballoon 608 (e.g., the outer surface of the cryoballoon 608) via any suitable mechanical method including, but not limited to, an adhesive, a polymer coating (e.g., as described above with respect to FIG. 4), and/or an outer balloon or sheath (e.g., as described above with respect to FIG. 5). In other embodiments, one or more microtubes 654 may be formed with and embedded in the cryoballoon 608.

Microtube 654 can extend distally and then proximally over the cryoballoon 608 such that a heated fluid may be continuously circulated through heating elements 636. For example, outer shaft 606 can also include a supply lumen 658 and a return lumen 660 for circulating a heated fluid through microtube 654. As shown in FIG. 6B, supply lumen 658 and return lumen 660 are each in fluid communication with opposing ends of lumen 656 of microtube 654. A heated fluid can be introduced through a supply port 662 of hub 616, which is in fluid communication with supply lumen 658, and the heated fluid can travel in a distal direction through catheter 600 via supply lumen 658 and into microtube 654. As the heated fluid travels over cryoballoon 608, the temperature of the heated fluid decreases. After traveling through microtube 654, the heated fluid can travel in a proximal direction through catheter 600 via return lumen 660. The heated fluid can exit catheter 600 via a return port 664 of hub 616, which is in fluid communication with return lumen 660. Accordingly, the heated fluid can flow through catheter 600 and microtube 654 in a circulating manner such that the temperature of the heated fluid within microtube 654 stays within a desired effective range.

A suitable configuration for the layout of supply lumen 658 and return lumen 660 is shown in FIG. 6A. It will be understood by those of ordinary skill in the art that other types of catheter construction are also amendable to the present technology, such as, without limitation thereto, a catheter shaft formed by various shafts extending therethrough for forming the lumens of the catheter. In addition, FIG. 6D illustrates an alternative configuration of the supply lumen and the return lumen. In FIG. 6D, outer shaft 606D includes guidewire shaft 628D and cryo-supply shaft 622D extending therethrough, as well as semicircular lumens 660D and 658D. The configuration of semicircular lumens 660D, 658D allows the heated fluid to extract heat from blood flow within the vessel lumen. Therefore, it may be desirable not to heat the heated fluid as much outside of the body as would otherwise be the case.

In addition, FIG. 6E illustrates an alternative microtube configuration that allows for the heated fluid to be continuously circulated therethrough. Instead of a microtube that extends distally and then proximally over the outer surface of the cryoballoon, microtube 654E includes first and second lumens 666, 668 for continuously circulating heated fluid through the heating elements. First lumen 666 can be placed in fluid communication with supply lumen 658 of outer shaft 606, and second lumen 668 can be placed in fluid communication with return lumen 660 of outer shaft 606. In addition, the distal ends of first lumen 666 and second lumen 668 can be in fluid communication with one another to allow the heated fluid to flow therebetween. For example, a heated fluid can travel in a distal direction through catheter 600 via supply lumen 658 and into first lumen 666 of microtube 654. The heated fluid can travel in a distal direction through first lumen 666 and then enter second lumen 668. The heated fluid can then travel in a proximal direction through second lumen 668 and continue to travel in a proximal direction through catheter 600 via return lumen 660. Accordingly, the dual lumens of microtubes 654E allow the heated fluid flow through catheter 600 and microtube 654 in a circulating manner such that the heated fluid within microtube 654 may stay at a steady or constant temperature. By utilizing dual lumens of the microtubes 654 to circulate the heated fluid, the microtubes may not need to extend distally and then proximally over the cryoballoon. In certain embodiments, a vacuum may be utilized to draw the spent heated fluid (i.e., proximally traveling fluid) out of the catheter. In other embodiments, the inward pumping pressure may force the fluid through the catheter.

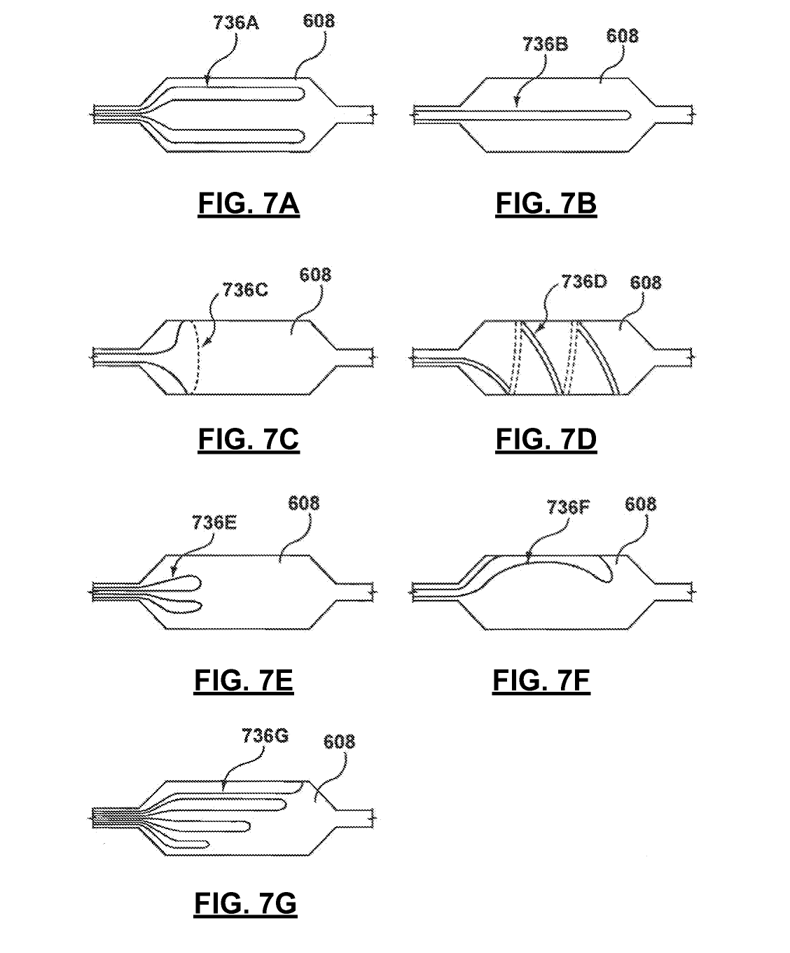

It will be apparent to those of ordinary skill in the art that various configurations of microtubes are possible in order to achieve a number of different ablation patterns. The path of heating elements 636 may extend in a spiral, a straight line, or partially around the circumference of cryoballoon 608. FIGS. 7A-7G illustrate various configurations of heating elements 736, wherein each configuration includes a microtube that extends distally and then proximally over the cryoballoon such that the configurations may be utilized to circulate heated fluid within single-lumen microtubes. However, if dual lumen microtubes are utilized (e.g., as described above with respect to FIG. 6E), the return path of the microtube can be omitted. FIG. 7A illustrates a plurality of heating elements 736A extending longitudinally along balloon 608, while FIG. 7B illustrates a single heating element 736B extending longitudinally along balloon 608. These microtube configurations can shield one or more linear or longitudinal strips of non-targeted tissue. FIG. 7C illustrates a single heating element 736C extending partially around a circumference of balloon 608. FIG. 7D illustrates a spiral heating element 736D extending around a circumference of balloon 608. FIG. 7G illustrates a plurality of straight heating elements 736G of differing lengths extending longitudinally along balloon 608. FIG. 7E is similar to FIG. 7G, except heating elements 736E are not of differing lengths and are restrained to a quadrant of balloon 608. FIG. 7F illustrates an arc or curved heating element 736F extending longitudinally along balloon 608. It will be apparent to those of ordinary skill in the art that numerous patterns or configurations of the heating elements 736 may be utilized in order to cause a desired ablation pattern having a combination of targeted and non-targeted tissue of the vessel wall.

Referring back to FIG. 6, catheter 600 can also include a thermocouple 638 that is similar to thermocouple 138 described above. If multiple microtubes 654 are utilized on cryoballoon 608, a separate thermocouple may be disposed adjacent to each microtube 654 for monitoring the temperature of tissue adjacent to the thermocouple at various locations on the device. As described herein, thermocouples 638 may be utilized in regulating or moderating the outer surface temperature of cryoballoon 608 because monitoring the temperature of cryoballoon 608 allows the operator to determine which heating elements should be active. With respect to microtube embodiments described herein, the heating elements 654 may be activated by selectively delivering the heated fluid therethrough and may be deactivated by stopping delivery of the heated fluid.

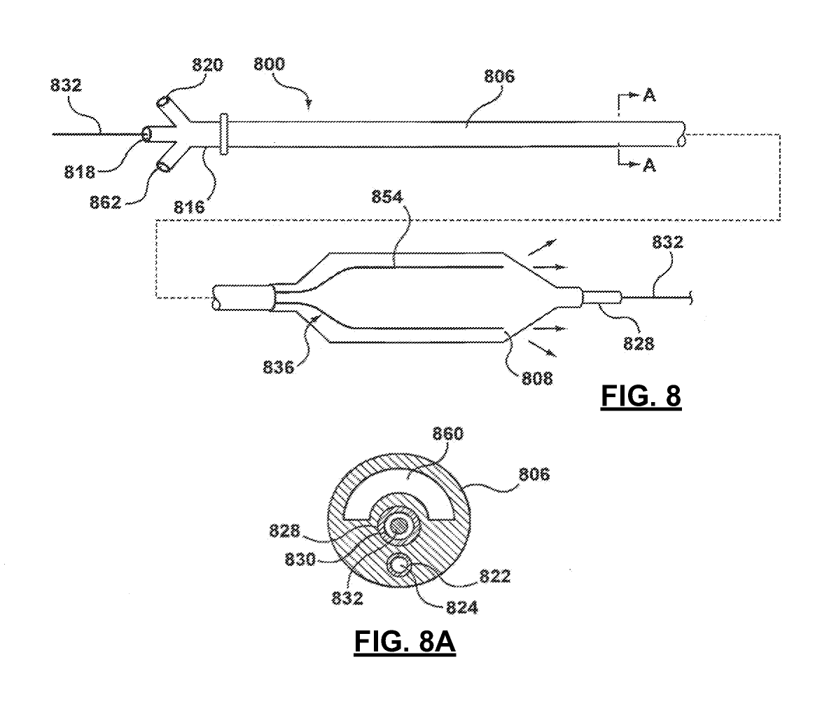

In another embodiment hereof, the heated fluid is not circulated through the microtube but rather is distally expelled into the bloodstream. Referring to FIG. 8 and FIG. 8A, a cryotherapy catheter 800 is utilized for ablating tissue to provide neuromodulation of the targeted nerves and heating elements 836 are utilized for shielding non-targeted tissue from ablation. Catheter 800 can include an outer shaft 806 with a cryoballoon 808 disposed at the distal end thereof. A guidewire shaft 828 and a cryo-supply shaft 822 can extend within a lumen of outer shaft 806. Similar to inner shaft 128 described above, guidewire shaft 828 can define a guidewire lumen 830 extending substantially an entire length of catheter 800 for accommodating a guidewire 832. Guidewire shaft 828 can have a proximal end (not shown) coupled to a proximal guidewire port 818 of a hub 816 and a distal end 832 terminating distally of cryoballoon 808 and defining a distal guidewire port. Cryo-supply shaft 822 can define an inflation lumen 824 and can have a distal end (not shown) that terminates within cryoballoon 808. A cryo-inflation port 820 of hub 816 can be placed in fluid communication with inflation lumen 824 of cryo-supply shaft 822, and cryo-supply shaft 822 can receive and deliver a cryogenic agent (e.g., N₂O liquid) into cryoballoon 808 as described above with respect to cryo-supply shaft 122 and cryoballoon 108.

Heating elements 836 can include two microtubes 854 which may be similar to microtubes 654 described above. Microtubes 854 can be tubular components disposed over cryoballoon 808 for receiving a heated fluid or gas. However, unlike microtube 654, microtubes 854 extend separately and distally over the cryoballoon 808 and the heated fluid exits the open distal ends of microtubes 854. Outer shaft 806 can include a supply lumen 860 in fluid communication with the proximal ends of the lumens (not shown) of microtubes 854. A heated fluid can be introduced through a supply port 862 of hub 816, which is in fluid communication with supply lumen 860, and the heated fluid can travel in a distal direction through catheter 800 via supply lumen 860 and into microtubes 854. The heated fluid can travel over cryoballoon 808 and exit from the distal ends of microtubes 854 such that the fluid is released into the blood stream. Thus, the heated fluid in this embodiment is biocompatible such that it can be released into the blood stream. For example, the heated fluid may include, saline, contrast media, plasma, and/or warmed gases (e.g., CO₂ or O₂). Accordingly, in this embodiment, the heated fluid flows through catheter 800 and microtube 854 in a non-circulating manner. Further, although heating elements 836 are shown as longitudinal, it will be apparent to those of ordinary skill in the art that other patterns, including the patterns shown in FIGS. 7A-7G (without return tubes), may be utilized to cause a desired ablation pattern having a combination of targeted and non-targeted tissue of the vessel wall. Although not shown, it will be understood that catheter 800 may also include one or more thermocouples as described above with respect to catheter 600 for regulating and/or moderating the outer surface temperature of cryoballoon 808.

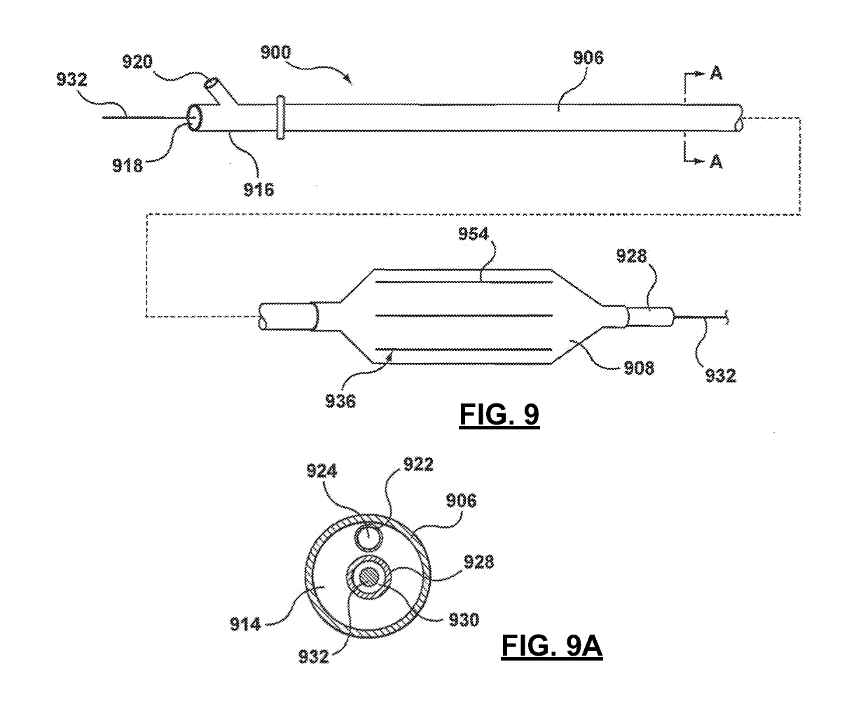

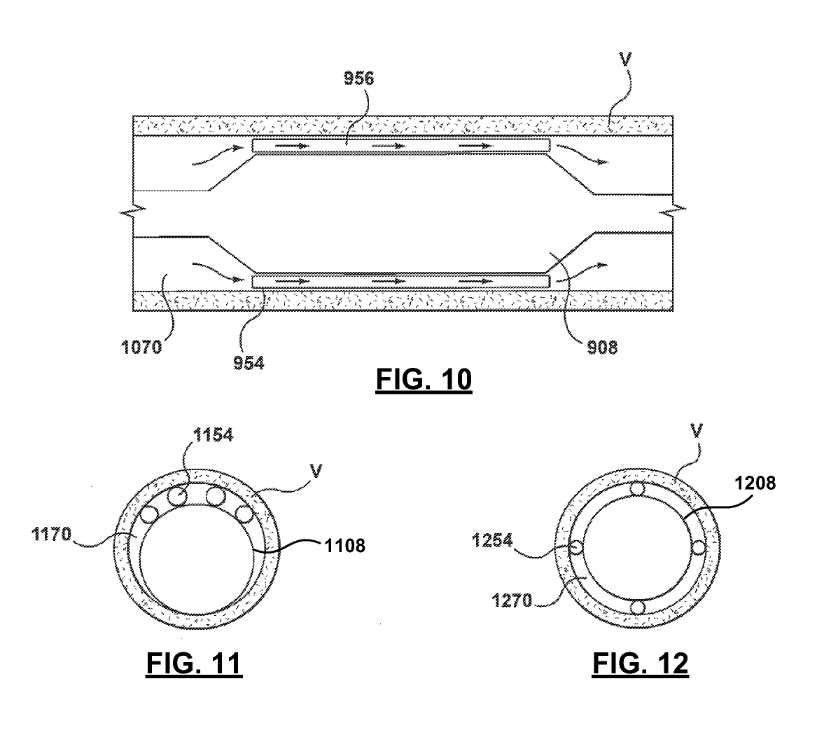

In yet another embodiment hereof, blood flow within the vessel can be utilized as the heated fluid through the microtubes to shield non-targeted tissue from ablation. By utilizing internal blood flow as the heated fluid that shields non-targeted tissue from ablation, the external heating system described above with respect to FIGS. 6-8A can be omitted. Referring to FIG. 9, FIG. 9A, and FIG. 10, a cryotherapy catheter 900 is utilized for ablating tissue to provide neuromodulation of the targeted nerves and heating elements 936 are utilized for shielding non-targeted tissue from ablation. Catheter 900 can include an outer shaft 906 with a cryoballoon 908 disposed at the distal end thereof. A guidewire shaft 928 and a cryo-supply shaft 922 can extend within a lumen 914 of outer shaft 906. Similar to inner shaft 128 described above, guidewire shaft 928 can define a guidewire lumen 930 extending substantially an entire length of catheter 900 for accommodating a guidewire 932. Guidewire shaft 928 can have a proximal end (not shown) coupled to a proximal guidewire port 918 of a hub 916 and a distal end 934 terminating distally of cryoballoon 908 and defining a distal guidewire port. Cryo-supply shaft 922 can define an inflation lumen 924 and has a distal end (not shown) that terminates within cryoballoon 908. A cryo-inflation port 920 of hub 916 can be placed in fluid communication with inflation lumen 924 of cryo-supply shaft 922, and cryo-supply shaft 922 can receive and deliver a cryogenic agent (e.g., N₂O liquid) into cryoballoon 908 as described above with respect to cryo-supply shaft 122 and cryoballoon 108.

Heating elements 936 can include a plurality of microtubes 954 which are similar to microtubes 654 described above. Microtubes 954 can be tubular components disposed over cryoballoon 908 for receiving a heated fluid or gas. However, unlike microtube 654, microtubes 954 extend only over the working length of cryoballoon 908 and have open proximal and distal ends. Cryoballoon 908 is shown disposed in a vessel V in FIG. 10. Blood flow from vessel lumen 1070 (see FIG. 10) enters into the proximal end of microtubes 954, flows through lumens 956 of microtubes 954 for shielding non-targeted tissue which is adjacent to the microtubes from ablation, and exits from the distal ends of microtubes 954 such that the blood returns to the blood stream. Accordingly, in this embodiment, the heated fluid is not delivered through the catheter, but rather blood flow is utilized as the heated fluid that protects non-targeted tissue from ablation. It will be apparent to those of ordinary skill in the art that lumens 956 of microtubes 954 can be sized to inhibit the blood from coagulating within or become otherwise impeding lumens 956. Further, although heating elements 936 are shown as longitudinal, it will be apparent to those of ordinary skill in the art that other patterns, such as the patterns shown in FIGS. 7A-7G (without return tubes), may be utilized to cause a desired ablation pattern having a combination of targeted and non-targeted tissue of the vessel wall.

Turning now to the cross-sectional view of FIG. 11, another embodiment utilizing blood flow to shield non-targeted tissue from ablation is shown. In FIG. 11, a cryoballoon 1108 has a plurality of microtubes 1154 disposed on the surface thereof. Cryoballoon 1108 is shown expanded in a vessel V. In this embodiment, cryoballoon 1108 is formed from a semi-compliant or noncompliant material. Balloons may be classified as being compliant, noncompliant or semi-compliant. Compliant balloons can be characterized by the balloon's ability to radially expand beyond its nominal diameter in response to increasing inflation pressure. Such balloons can be said to follow a stress-strain curve obtained by plotting balloon diameter versus inflation pressure. Noncompliant balloons can be characterized by a nearly flat stress-strain curve illustrating that the balloon diameter expands very little over the range of usable inflation pressures. In one embodiment, cryoballoon 1108 can be 10% or less compliant and may be formed from nylon.

Microtubes 1154 can be solid tubular components formed of an insulative material, such as nylon, PEBAX polymer, and/or silicone. Microtubes 1154 can be effective for spacing a portion of cryoballoon 1108 away from the vessel wall. In addition, since cryoballoon 1108 is formed from a semi-compliant or non-compliant material, cryoballoon 1108 does not expand into the spaces between microtubes 1154. Rather, blood flow from vessel lumen 1170 flows between and around microtubes 1154, such that microtubes 1154 essentially create a blood flow path for shielding non-targeted tissue from ablation. Tissue which is adjacent to microtubes 1154 and blood flow is shielded or protected from ablation. Thus, in the embodiment of FIG. 11, approximately the top half of the vessel wall is shielded from ablation, and the ablation pattern from cryoballoon 1108 is approximately the bottom half of the vessel wall which contacts and abuts against cryoballoon 1108. Further, it will be apparent to those of ordinary skill in the art that other patterns of microtubes 1154 may be utilized to cause a desired ablation pattern having a combination of targeted and non-targeted tissue of the vessel wall.

Conversely, solid microtubes may be utilized in such as way that they cause ablation of the vessel. Referring to the embodiment shown in FIG. 12, a cryoballoon 1208 can have a plurality of solid tubular microtubes 1254 disposed on the surface thereof. Cryoballoon 1208 is shown expanded in a vessel V and is formed from a semi-compliant or noncompliant material as described above with respect to FIG. 11. Microtubes 1254 can be formed of a conductive material, such as metals (e.g., aluminum, stainless steel, and cobalt chromium), glass fibers (e.g., fiber optics), and/or polymers loaded with conductive fillers (e.g., the above-mentioned materials, brass, copper, barium sulphate, or glass). The conductive material of microtubes 1254 may be a continuous single strand of material or may be a multi-strand structure for added flexibility. Microtubes 1254 can space a portion of cryoballoon 1208 away from the vessel wall and, since cryoballoon 1208 is formed from a semi-compliant or non-compliant material, cryoballoon 1208 does not expand into the spaces between microtubes 1254. In this embodiment, however, conductive microtubes 1254 transfer the cryotherapeutic cooling from cryoballoon 1208 to the vessel wall. Tissue which is adjacent to cryoballoon 1208 and/or microtubes 1254 is thus ablated. Blood flow from vessel lumen 1270 can flow between and around microtubes 1254, and tissue adjacent to the blood flow may be shielded or protected from ablation. Thus, in the embodiment of FIG. 12, the resulting ablation pattern includes four linear or longitudinal strips of tissue adjacent to microtubes 1254 with the areas of tissue between the microtubes 1254 being shielded from ablation by blood flow. Further, it will be apparent to those of ordinary skill in the art that other patterns of microtubes 1254 may be utilized to cause a desired ablation pattern having a combination of targeted and non-targeted tissue of the vessel wall.

### EXAMPLES

1. A cryotherapeutic device, comprising:

an elongated shaft having a proximal portion and a distal portion, wherein the shaft is configured to locate the distal portion at a treatment site in a renal vessel;

a cryoballoon affixed at the distal portion, the cryoballoon being configured to apply therapeutically-effective cooling to ablate tissue of a wall of the renal vessel; and

a plurality of heating elements arranged about the cryoballoon, wherein the plurality of heating elements are individually controllable to selectively deliver heat to tissue of a wall of the renal vessel proximate the cryoballoon.

2. The cryotherapeutic device of example 1 wherein the plurality of heating elements is a plurality of individual electrodes, each individual electrode being electrically coupled to a power source at the proximal portion of the shaft via a corresponding wire extending along the shaft.

3. The cryotherapeutic device of example 1 wherein the plurality of heating elements is a plurality of individual microtubes, each individual microtube including at least one lumen configured for receiving a heated fluid.

4. The cryotherapeutic device of example 1, further comprising a plurality of thermocouples at the distal portion of the shaft, wherein the thermocouples are configured to monitor temperatures at the cryoballoon.

5. The cryotherapeutic device of example 4 wherein each thermocouple is adjacent to a corresponding heating element.

6. The cryotherapeutic device of example 1, wherein the plurality of heating elements is configured to selectively deliver thermal energy to an outer surface of the cryoballoon, the thermal energy having a temperature between about 5° C. and about 45° C.

7. A cryotherapeutic device, comprising:

an elongated shaft having a distal portion, the shaft being configured to locate the distal portion in a vessel;

a cryoballoon affixed to the distal portion, the cryoballoon having an expanded configuration; and

a microtube arranged on the cryoballoon, the microtube having a lumen configured to receive a heated fluid, wherein the microtube is configured to be positioned between the cryoballoon and a vessel wall of the vessel when the cryoballoon is in the expanded configuration.

8. The cryotherapeutic device of example 7 wherein:

the shaft includes a supply lumen and a return lumen, the supply lumen being configured to deliver heated fluid to the microtube, and the return lumen being configured to receive heated fluid from the microtube; and

the lumen of the microtube includes a first end portion in fluid communication with the supply lumen and a second end portion in fluid communication with the return lumen such that the heated fluid circulates through the microtube.

9. The cryotherapeutic device of example 7 wherein:

the shaft includes a supply lumen and a return lumen, the supply lumen being configured to deliver heated fluid to the microtube, and the return lumen being configured to receive heated fluid from the microtube;

the lumen of the microtube is a first lumen in fluid communication with the supply lumen; and

the microtube further comprises a second lumen in fluid communication with the return lumen, the first and second lumens being configured to circulate the heated fluid through the microtube.

10. The cryotherapeutic device of example 7 wherein:

the shaft includes a supply lumen configured to deliver heated fluid to the microtube; and

the microtube includes a proximal end portion in fluid communication with the supply lumen and a distal end portion open to the vessel such that the microtube is configured to expel the heated fluid into the vessel.

11. The cryotherapeutic device of example 7 wherein the microtube includes an open proximal end portion and an open distal end portion, and wherein the open proximal and distal end portions are configured to be in fluid communication with a blood stream of the vessel such that the heated fluid is blood.

12. The cryotherapeutic device of example 7 wherein:

the microtube is a solid shaft configured to space a portion of the cryoballoon away from the vessel wall when the cryoballoon is in the expanded configuration; and

the heated fluid is blood that flows through the vessel around the microtube.

13. The cryotherapeutic device of example 12 wherein the cryoballoon comprises a semi-compliant and/or a noncompliant material.

14. The cryotherapeutic device of example 12 wherein the microtube comprises an insulative material.

15. The cryotherapeutic device of example 12 wherein the microtube comprises a conductive material configured to transfer cryotherapeutic cooling from the cryoballoon to the vessel wall.

16. A method of treating a human patient, the method comprising:

locating a distal portion of an elongated shaft within a renal vessel of the patient;

delivering refrigerant to a cryoballoon affixed the distal portion of the shaft, wherein the cryoballoon includes at least one heating element arranged about the cryoballoon to contact a wall of the renal vessel when the cryoballoon is in an expanded configuration in the renal vessel;

expanding the refrigerant within the cryoballoon to cool the cryoballoon;

cryogenically ablating targeted tissue of the vessel wall proximate to an outer surface of the cryoballoon; and

transferring heat to non-targeted tissue of the vessel wall proximate the at least one heating element to inhibit cryogenic ablation of the non-targeted tissue.

17. The method of example 16 wherein transferring heat to non-targeted tissue of the vessel wall proximate the at least one heating element comprises transferring heat to the non-targeted tissue via an electrical current delivered to a plurality of electrodes at an outer surface of the cryoballoon.

18. The method of example 17, further comprising:

measuring temperatures proximate the plurality of electrodes via adjacent thermocouples; and

independently controlling the individual electrodes to selectively transfer the heat to the non-targeted tissue in response to the measured temperatures.

19. The method of example 16 wherein transferring heat to non-targeted tissue of the vessel wall proximate the at least one heating element comprises receiving a heated fluid in a plurality of lumens defined a plurality of microtubes.

20. The method of example 19, further comprising circulating the heated fluid across a length of the cryoballoon during cryogenic ablation of the targeted tissue.

21. The method of example 19 wherein:

receiving the heated fluid comprises receiving the heated fluid from a supply lumen in the shaft; and

the method further comprises distally dispelling the heated fluid into a blood stream of the renal vessel.

22. The method of example 19 wherein:

receiving the heated fluid comprises receiving blood from a blood stream of the renal vessel at proximal openings of the lumens; and

the method further comprises distally dispelling the blood into the blood stream via distal openings of the lumens.

23. The method of example 16, further comprising:

measuring a temperature of an outer surface of the cryoballoon;

selectively increasing the temperature of the outer surface via the at least one heating element when the measured temperature is above a threshold temperature.

24. The method of example 16 wherein transferring heat to non-targeted tissue of the vessel wall proximate the at least one heating element comprises maintaining temperatures of non-targeted tissue proximate the at least one heating element between 5° C. and 45° C. during cryogenic ablation of the targeted tissue.

### CONCLUSION

While various embodiments according to the present technology have been described above, it should be understood that they have been presented by way of illustration and example only, and not limitation. It will be apparent to persons skilled in the relevant art that various changes in form and detail can be made therein without departing from the spirit and scope of the present technology. Thus, the breadth and scope of the present technology should not be limited by any of the above-described embodiments. It will also be understood that each feature of each embodiment discussed herein, and of each reference cited herein, can be used in combination with the features of any other embodiment. All patents and publications discussed herein are incorporated by reference herein in their entirety.

Where the context permits, singular or plural terms may also include the plural or singular terms, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout the disclosure to mean including at least the recited feature(s) such that any greater number of the same feature(s) and/or additional types of other features are not precluded. It will also be appreciated that various modifications may be made to the described embodiments without deviating from the present technology. Further, while advantages associated with certain embodiments of the present technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

I/We claim:

1. A cryotherapeutic device, comprising:
an elongated shaft having a proximal portion and a distal portion, wherein the shaft is configured to locate the distal portion at a treatment site in a renal vessel;
a cryoballoon affixed at the distal portion, the cryoballoon being configured to apply therapeutically-effective cooling to ablate tissue of a wall of the renal vessel; and
a plurality of heating elements arranged about to the cryoballoon, wherein the plurality of heating elements are individually controllable to selectively deliver heat to tissue of a wall of the renal vessel proximate the cryoballoon.

2. The cryotherapeutic device of claim 1 wherein the plurality of heating elements is a plurality of individual electrodes, each individual electrode being electrically coupled to a power source at the proximal portion of the shaft via a corresponding wire extending along the shaft.

3. The cryotherapeutic device of claim 1 wherein the plurality of heating elements is a plurality of individual microtubes, each individual microtube including at least one lumen configured for receiving a heated fluid.

4. The cryotherapeutic device of claim 1, further comprising a plurality of thermocouples at the distal portion of the shaft, wherein the thermocouples are configured to monitor temperatures at the cryoballoon.

5. The cryotherapeutic device of claim 4 wherein each thermocouple is adjacent to a corresponding heating element.

6. The cryotherapeutic device of claim 1, wherein the plurality of heating elements is configured to selectively deliver thermal energy to an outer surface of the cryoballoon, the thermal energy having a temperature between about 5° C. and about 45° C.

7. A cryotherapeutic device, comprising:
an elongated shaft having a distal portion, the shaft being configured to locate the distal portion in a vessel;
a cryoballoon affixed to the distal portion, the cryoballoon having an expanded configuration; and
a microtube arranged on the cryoballoon, the microtube having a lumen configured to receive a heated fluid, wherein the microtube is configured to be positioned between the cryoballoon and a vessel wall of the vessel when the cryoballoon is in the expanded configuration.

8. The cryotherapeutic device of claim 7 wherein:
the shaft includes a supply lumen and a return lumen, the supply lumen being configured to deliver heated fluid to the microtube, and the return lumen being configured to receive heated fluid from the microtube; and
the lumen of the microtube includes a first end portion in fluid communication with the supply lumen and a second end portion in fluid communication with the return lumen such that the heated fluid circulates through the microtube.

9. The cryotherapeutic device of claim 7 wherein:
the shaft includes a supply lumen and a return lumen, the supply lumen being configured to deliver heated fluid to the microtube, and the return lumen being configured to receive heated fluid from the microtube;
the lumen of the microtube is a first lumen in fluid communication with the supply lumen; and
the microtube further comprises a second lumen in fluid communication with the return lumen, the first and second lumens being configured to circulate the heated fluid through the microtube.

10. The cryotherapeutic device of claim 7 wherein:
the shaft includes a supply lumen configured to deliver heated fluid to the microtube; and
the microtube includes a proximal end portion in fluid communication with the supply lumen and a distal end portion open to the vessel such that the microtube is configured to expel the heated fluid into the vessel.

11. The cryotherapeutic device of claim 7 wherein the microtube includes an open proximal end portion and an open distal end portion, and wherein the open proximal and distal end portions are configured to be in fluid communication with a blood stream of the vessel such that the heated fluid is blood.

12. The cryotherapeutic device of claim 7 wherein:
the microtube is a solid shaft configured to space a portion of the cryoballoon away from the vessel wall when the cryoballoon is in the expanded configuration; and
the heated fluid is blood that flows through the vessel around the microtube.

13. The cryotherapeutic device of claim 12 wherein the cryoballoon comprises a semi-compliant and/or a noncompliant material.

14. The cryotherapeutic device of claim 12 wherein the microtube comprises an insulative material.

15. The cryotherapeutic device of claim 12 wherein the microtube comprises a conductive material configured to transfer cryotherapeutic cooling from the cryoballoon to the vessel wall.

16. A method of treating a human patient, the method comprising:
locating a distal portion of an elongated shaft within a renal vessel of the patient;
delivering refrigerant to a cryoballoon affixed the distal portion of the shaft, wherein the cryoballoon includes at least one heating element arranged about the cryoballoon to contact a wall of the renal vessel when the cryoballoon is in an expanded configuration in the renal vessel;
expanding the refrigerant within the cryoballoon to cool the cryoballoon;
cryogenically ablating targeted tissue of the vessel wall proximate to an outer surface of the cryoballoon; and
transferring heat to non-targeted tissue of the vessel wall proximate the at least one heating element to inhibit cryogenic ablation of the non-targeted tissue.

17. The method of claim 16 wherein transferring heat to non-targeted tissue of the vessel wall proximate the at least one heating element comprises transferring heat to the non-targeted tissue via an electrical current delivered to a plurality of electrodes at an outer surface of the cryoballoon.

18. The method of claim 17, further comprising:
measuring temperatures proximate the plurality of electrodes via adjacent thermocouples; and
independently controlling the individual electrodes to selectively transfer the heat to the non-targeted tissue in response to the measured temperatures.

19. The method of claim 16 wherein transferring heat to non-targeted tissue of the vessel wall proximate the at least one heating element comprises receiving a heated fluid in a plurality of lumens defined a plurality of microtubes.

20. The method of claim 19, further comprising circulating the heated fluid across a length of the cryoballoon during cryogenic ablation of the targeted tissue.

21. The method of claim 19 wherein:
receiving the heated fluid comprises receiving the heated fluid from a supply lumen in the shaft; and
the method further comprises distally dispelling the heated fluid into a blood stream of the renal vessel.

22. The method of claim 19 wherein:
receiving the heated fluid comprises receiving blood from a blood stream of the renal vessel at proximal openings of the lumens; and
the method further comprises distally dispelling the blood into the blood stream via distal openings of the lumens.

23. The method of claim 16, further comprising:
measuring a temperature of an outer surface of the cryoballoon;
selectively increasing the temperature of the outer surface via the at least one heating element when the measured temperature is above a threshold temperature.

24. The method of claim 16 wherein transferring heat to non-targeted tissue of the vessel wall proximate the at least one heating element comprises maintaining temperatures of non-targeted tissue proximate the at least one heating element between 5° C. and 45° C. during cryogenic ablation of the targeted tissue.
